(19) United States
(12) Patent Application Publication
(10) Pub. No.: US 2014/0276644 A1
(43) Pub. Date: Sep. 18, 2014

(54) Elastic Catheter

(71) Applicant: HLT, Inc., Maple Grove, MN (US)

(72) Inventor: Dale K. Nelson, Inver Grove Heights, MN (US)

(73) Assignee: HLT, Inc., Maple Grove, MN (US)

(21) Appl. No.: 14/213,920

(22) Filed: Mar. 14, 2014

Related U.S. Application Data
(60) Provisional application No. 61/786,532, filed on Mar. 15, 2013.

(51) Int. Cl. A61M 25/00; A61F 2/24

(52) U.S. Cl. CPC A61M 25/005; A61M 25/008; A61F 2/2427
USPC 604/527; 604/526

(57) ABSTRACT

A catheter with an elastic reinforcement layer allows its catheter tube to expand from a native diameter to an expanded diameter. In this respect, a device or implant with a larger diameter than that of the catheter tube's native diameter size can be passed through the catheter without damage.

### RELATED APPLICATIONS

This application claims priority to U.S. Provisional Application Ser. No. 61/786,532 filed Mar. 15, 2013 entitled Expandable Catheter, which is hereby incorporated herein by reference in its entirety.

### BACKGROUND OF THE INVENTION

Intravascular catheters can be used for a variety of different purposes, such as delivering prosthetic devices or providing therapeutic treatment to various locations in a patient's body. Since these catheters must travel through tortuous pathways of variously-sized vessels, they are constructed so as to allow some bending and torque transmission without kinking, bursting, breaking apart, or otherwise substantially changing shape. Typically, these properties are achieved by including a reinforcement layer within the catheter in the form of a coil or braid, using materials such as stainless steel, Kevlar, Dacron, liquid crystal polymer, or materials of similar characteristics.

### SUMMARY OF THE INVENTION

One embodiment according to the present invention is directed to a catheter with an elastic reinforcement layer which allows its catheter tube to expand from a native diameter to an expanded diameter. In this respect, a device or implant with a larger diameter than that of the catheter tube's native diameter size can be passed through the catheter without damage.

In one embodiment, the catheter comprises a reinforcement layer that includes a plurality of braided, shape-memory wires. In another embodiment, the reinforcement layer can include both braided, superelastic wires and an elastic material. The reinforcement layer can extend the entire length of the catheter tube or can terminate prior to either the proximal or distal ends.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a side view of a catheter with an elastic reinforcement layer;

FIGS. 2-3 are partial cut-away views of the catheter of FIG. 1;

FIG. 4 is a cross sectional view of a catheter tube with an elastic reinforcement layer; and,

FIG. 5 is a partial cut-away view of the catheter tube from FIG. 4.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

FIGS. 1, 2, and 3 illustrate an intravascular catheter 100 with an elastic reinforcement layer that allows the catheter tube 104 to radially expand when a large implant 108 or a similar device is moved through it. Once the implant 108 has passed, the tube 104 can contract back to its native diameter. In this regard, a physician may select a generally smaller diameter catheter for a procedure than would otherwise be selected, while allowing devices/implants 108 larger than the catheter's native diameter to also be passed through. Additionally, the elastic nature of the catheter 100 prevents these larger devices/implants 108 from damaging the catheter 100 or from being damaged themselves.

In one embodiment seen in FIGS. 4 and 5, the catheter tube 104 is at least composed of an outer layer 110, an elastic reinforcement layer 112, and an inner layer 114. In one example, the reinforcement layer is composed of a plurality of braided, woven, or coiled wires made of one or more super-elastic shape-memory materials. Example braids for catheter reinforcement could be constructed from 16 to 48 wires, with a range of patterns to provide the reinforcement layer. Examples of braid patterns include a diamond pattern half-load (one wire over one, under one), a diamond pattern full load (two wires under two, over two), or regular braid pattern (one wire under two, over two). See Figures attached. Picks per inch (PPI) could be in the range of 50-100 PPI. Examples of such shape-memory material include: nickel titanium (also known as Nitinol), cobalt-chromium, titanium-palladium-nickel, nickel-titanium-copper, gold-cadmium, iron-zinc-copper-aluminum, titanium-niobium-aluminum, hafnium-titanium-nickel, iron-manganese-silicon, tantalum, shape-memory polyurethanes, and any other shape-memory metal, alloy, or polymer that is known in the art. In another example, the shape-memory wire can have a round diameter or can be a rectangular, ribbon shape. In another example, the elastic reinforcement layer 112 comprises a combination of braided, shape-memory wires and a second elastomer layer, such as elastic fibers interwoven within the superelastic braid. Elastomers in this application do not require high elongation and could include nylon, polyurethane, polyester, polyolefin blends, and styrenic block copolymers.

In another example, the reinforcement layer 112 is biased or memory set (e.g., heat set) to a native or default diameter that allows for further expansion. For example, the reinforcement layer 112 braided and memory set to a diameter of about 0.200 inches and can further expand to a diameter of about 0.250 inches to accommodate a large implant.

Preferably, the inner layer 114 and outer layer 110 allow or accommodate the expansion of the reinforcement layer 112. In one example, both layers 110 and 114 are composed of an elastic polymer that stretches and compresses/retracts along with the reinforcement layer 114. Examples of such material include polyetheramide (pebax), other nylons, polyurethanes, and fluoropolymers (PTFE, FEP, etc.). Preferably, the inner layer 114 is either composed of a low friction material or includes a low friction coating to facilitate movement of devices/implants through the catheter tube 104. In another example, the inner layer 114 and outer layer 112 are composed of a non-compliant or semi-compliant material that folds or “bunches” in one or more areas in the native diameter and opens or spreads out when expanded by a device/implant.

Preferably, the catheter tube 104 includes one or more radiopaque marker rings 116 composed of a radiopaque material such as platinum or titanium, as seen in FIG. 4. In the example of FIG. 4, the ring 116 includes a diagonal cut 116A which allows the ring to open or separate as a larger device or implant is passed through. In other words, the ring 116 has a generally “C” shape. In another example, the radiopaque marker can be a coil. The reinforcement layer 112 may also further include one or more radiopaque wires woven amongst the shape-memory wires to provide a physician with further visual cues during a procedure.

In one embodiment, the reinforcement layer 112 extends entirely between the proximal and distal end of the catheter tube 104. In another embodiment, the reinforcement layer 112 extends only partially along the length of the catheter tube 104. In another example, the reinforcement layer 112 extends from a proximal end of the catheter tube 104 to a location between about 1 mm to 10 mm from the distal tip, such that no further reinforcement is present near the distal end. In another example embodiment, the reinforcement layer 112 is only extend from the distal end of the tube 104 to a location between about 1 mm to 10 mm proximally.

In one example operational use of the catheter, a distal end of a guidewire is advanced near a target location within a patient and the catheter 100 is advanced over the guidewire. The guidewire is removed and an implant delivery device 106 (e.g., prosthetic heart valve delivery device) is advanced into the catheter's hub 102. Since the implant 108 of the delivery device 106 is somewhat larger than the catheter tube 104, the reinforcement layer 112, as well as layers 110 and 114, expand outwardly at region 104A. As the implant 108 advances down the catheter tube 104, it expands distal portions of the tube 104 while proximal portions of the tube 104 compress or return to its native diameter. Finally, the implant 108 is advance out of the distal end of the catheter tube 104 as seen in FIG. 3.

In some circumstances, the implant 108 (or device) must be retracted back into the catheter. Since prior art catheters tend not to expand, this retraction or retrieval can exert unwanted force on the implant or device. However, since the present catheter 100 can expand, these retrieval forces are reduced as compared to a similarly-sized catheter. Hence, the present catheter 100 may provide a gentler retraction process.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A catheter comprising:
a catheter hub; and,
a catheter tube having a proximal end connected to said catheter hub; said catheter tube elastically expanding from a predetermined, native diameter to accommodate passage of an implant having an implant diameter larger than said predetermined, native diameter.

2. The catheter of claim 1, wherein said catheter tube comprises a reinforcement layer comprising braided, shape-memory wires.

3. The catheter of claim 2, wherein said shape-memory wires form a tubular shape that is shape-memory set to expand to said native diameter.

4. The catheter of claim 3, wherein said reinforcement layer extends between said proximal end of said catheter tube and a distal end of said catheter tube.

5. The catheter of claim 3, wherein said reinforcement layer extends between said proximal end of said catheter tube and a location proximal of a distal end of a said catheter tube.

6. The catheter of claim 5, wherein said location proximal of said distal end of said catheter tube is within a range of about 1 mm to 10 mm.

7. The catheter of claim 1, wherein said catheter tube comprises a reinforcement layer comprising a shape-memory wire arranged in a coil shape.

8. A catheter comprising:
a catheter tube forming a passage opening a proximal end and a distal end; said catheter tube being elastically expandable from a predetermined, native diameter to accommodate passage of a medical device having a device diameter larger than said predetermined, native diameter.

9. The catheter of claim 8, wherein said catheter tube comprises a reinforcement layer comprising braided, shape-memory wires.

10. The catheter of claim 9, wherein said catheter tube further comprises an outer layer disposed over said reinforcement layer and an inner layer disposed within said reinforcement layer; said outer layer and said inner layer being composed of a compliant material that expandable with said reinforcement layer.

11. The catheter of claim 8, wherein said catheter tube comprises a reinforcement layer comprising a coiled, shape-memory wire.

12. The catheter of claim 8, wherein said catheter tube further comprises a radiopaque marker having a generally “C” shape.

13. The catheter of claim 8, wherein said catheter tube further comprises a radiopaque marker having a coil shape.

14. The catheter of claim 9, wherein said reinforcement layer extends between a proximal end of said catheter tube and a distal end of said catheter tube.

15. The catheter of claim 9, wherein said reinforcement layer extends between a proximal end of said catheter tube and a location proximal of a distal end of a said catheter tube.

16. The catheter of claim 9, wherein said shape-memory wires form a tubular shape that is shape-memory set to expand to said native diameter.

17. The catheter of claim 9, wherein said braided, shape-memory wires are composed of Nitinol.

18. A method of using a catheter comprising:
providing a catheter having a native diameter;
providing a medical device having a device diameter larger than said native diameter;
advancing said medical device into said catheter so as to cause said catheter to elastically expand around said medical device.

19. The method of claim 18, further comprising returning said catheter to said native diameter.

20. The method of claim 18, wherein advancing said medical device into said catheter further comprises expanding braided reinforcement layer within said catheter.
